(19) United States
(12) Patent Application Publication
(10) Pub. No.: US 2023/0130301 A1
(43) Pub. Date: Apr. 27, 2023

(54) Intubation Protective Enclosure and Method of Use and Disinfecting

(71) Applicants: Howards J SIEGEL, Sands Points, NY (US); Christopher BURMESTER, Verona, NJ (US); Drexel University, Philadelphia, PA (US); The Trustees of the University of Pennsylvania, Philadelphia, PA (US)

(72) Inventors: Kara L. Spiller, Glenside, PA (US); Sharon Walker, Narberth, PA (US); Caroline Schauer, Hulmeville, PA (US); Amy Throckmorton, Cherry Hill, PA (US); Ian Marcus, Philadelphia, PA (US); Alexander V. Dudchenko, San Jose, CA (US); Michael Kohanski, Wynnewood, PA (US); Christopher Rassekh, Lafayette Hill, PA (US); Tiffany Chao, Philadelphia, PA (US); Elizabeth R. Kahle, Philadelphia, PA (US); Michael Waring, Philadelphia, PA (US); Liang Chung James Lo, Devon, PA (US); Michele Marcolongo, Aston, PA (US); Howard J. Siegel, Sands Point, NY (US); Christopher Burmester, Verona, NJ (US); Heather Smith, Riverside, CA (US)

(21) Appl. No.: 17/914,430

(22) PCT Filed: Mar. 26, 2021
(86) PCT No.: PCT/US2021/024344

§ 371 Date: Sep. 26, 2022

Related U.S. Application Data
(60) Provisional application No. 63/000,106, filed on Mar. 26, 2020.
(60) Provisional application No. 63/012,746, filed on Apr. 20, 2020.
(60) Provisional application No. 63/077,655, filed on Sep. 13, 2020.

(51) Int. Cl. A61G 10/00

(52) U.S. Cl. CPC A61G 10/005

(57) ABSTRACT

The current invention provides an enclosure that can be placed over a patient in order to contain bodily fluids. The device includes a clear plastic sheet that fits snugly over a frame. The sides of the device contain access ports for physicians or their instruments to enter the patient’s area. These ports may be holes or hanging strips of sheet that overlap so that they form a sufficient seal while also allowing easy access to the patient when pushed aside. The bottoms of the sheet may contain weights to hold the sheet down. There is the possibility of including filters or positive/negative air flow ports. The entire device may be disposable. The frame may be solid, or the frame may pop open like an enclosure or umbrella. The device may also have straps that can be tightened underneath the bed or stretcher to secure the entire device in place.

### CROSS-REFERENCE TO RELATED APPLICATION

The present application claims the benefit of U.S. Provisional Pat. Application Serial No. 63/000,106, filed on Mar. 26, 2020; U.S. Provisional Pat. Application Serial No. 63/004,907, filed on Apr. 3, 2020; U.S. Provisional Pat. Application Serial No.63/012,746, filed on Apr. 20, 2020; and U.S. Provisional Pat. Application Serial No. 63/077,655, filed on Sep. 13, 2020; all of which are incorporated herein by reference in their entireties.

### BACKGROUND OF THE INVENTION

### Field of the Invention

The invention relates to a portable enclosure that can be placed over a patient’s head and chest area during intubation or other respiratory-related procedure to prevent or otherwise restrict the flow of exhaled gases, fluids, and particles from the patient into the surrounding environment.

### Description of the Related Art

The COVID-19 pandemic has heightened the awareness of infected people exhaling contaminated air into their immediate airspace, potentially infecting those around them. This situation is especially acute in the medical field, where clinicians often are required to treat patients with respiratory issues, such as via intubation, requiring the clinicians to be in close contact with a patient’s mouth and nose.

It would be beneficial to provide a protective enclosure between an infected patient and clinicians to reduce the exposure of contaminated air exhaled from the patient by the clinicians. Further, it would be beneficial to provide a protective enclosure that can be used in hospital, clinical, and field environments.

### BRIEF SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a containment enclosure comprising a frame having a rear portion, a left portion attached to the rear portion, a right portion attached to the rear portion, opposite from the left portion, and a top portion attached to the rear portion, the left portion, and the right portion. A flexible transparent sheet is disposed over at least part of the frame and covers a front portion.

In an alternative embodiment, the present invention is a contaminant enclosure comprising a frame sized to extend over the head and torso of an adult human. The frame has an upper perimeter and a lower perimeter, a transparent flexible material attached to the upper perimeter of the frame, and a flexible material extending at least partially around the lower perimeter. The flexible material incudes a plurality of vertically hanging strips.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a right front perspective view of an enclosure according to an exemplary embodiment of the present invention;

FIG. 2 is a left rear perspective view of the enclosure of FIG. 1;

FG. 3 is a right rear perspective view of the enclosure of FIG. 1;

FIG. 4 is a left front perspective view of the enclosure of FIG. 1 (with patient removed);

FIG. 5 is a top plan view of a flexible material used with the enclosure of FIG. 1;

FIG. 6 is a side elevational view of a wall of the enclosure of FIG. 1, showing optional gloves attached to the side wall of the enclosure;

FIG. 7 is a flowchart to use to determine whether the enclosure of FIGS. 1-6 should be used;

FIG. 8 illustrates the step of placing the enclosure of FIG. 1 over the head of a patient;

FIG. 9 illustrates the step of securing straps of the enclosure of FIG. 1 underneath a bed on which the patient is lying;

FIG. 10 illustrates the step of tucking in sides of the sheet of the enclosure of FIG. 1 under the patient;

FIG. 11 illustrates the step of connecting suction tubing to the sheet of the enclosure of FIG. 1;

FIG. 12 illustrates the step of a clinician working on a patient through the enclosure of FIG. 1;

FIG. 13 shows an exemplary graph of the amount of time required to draw air from inside the enclosure of FIG. 1 to achieve approximately 99% removal of potentially contaminated air from inside enclosure as a function of the exhaust air flow rate;

FIG. 14 illustrates the removal of tubing from the enclosure of FIG. 1;

FIG. 15A illustrates the removal of straps from the enclosure of FIG. 1;

FIG. 15B illustrates removal of the enclosure of FIG. 1 from a patient;

FIG. 16 is a rear perspective view of an enclosure according to an alternative exemplary embodiment of the present invention;

FIG. 17 is a front elevational view of an enclosure according to another alternative exemplary embodiment of the present invention;

FIG. 18 is a rear elevational view of the enclosure of FIG. 17;

FIG. 19 is a side elevational view of the enclosure of FIG. 17;

FIG. 20 is a top plan view of the enclosure of FIG. 17;

FIG. 21 is a top plan view of the sides of the enclosure of FIG. 17;

FIG. 22 is a top plan view of a flexible material draped over the sides of the enclosure of FIG. 17;

FIG. 23 is a perspective view of a first side of the enclosure of FIG. 17 being raised;

FIG. 24 is a perspective view of the second side of the enclosure of FIG. 17 being raised;

FIG. 25 is a perspective view of the flexible material being put into place on the second side of the enclosure;

FIG. 26 is a perspective view of both sides of the enclosure having been raised;

FIG. 27 is a perspective view of the second side being locked into place;

FIG. 28 is a perspective view showing the flexible material being attached to the first side;

FIG. 29 is a rear perspective view of an enclosure according to still another exemplary embodiment of the present invention;

FIG. 30 is an enlarged left side elevational view of a roof clamp of the enclosure of FIG. 28;

FIG. 31 is a top perspective view of the enclosure of FIG. 29;

FIG. 32 is an enlarged right side elevational view of the enclosure of FIG. 29;

FIG. 33 is a top plan view of the enclosure of FIG. 29 in a collapsed/storage condition;

FIG. 34 is a front perspective view of the enclosure of FIG. 29 showing optional through-openings and equipment attached to the panels and roof of the enclosure of FIG. 29;

FIG. 35 is a top plan view of the enclosure of FIG. 29 used with a patient;

FIG. 36 is a schematic drawing of a test set-up used for aerosol evaluation testing;

FIG. 37A is a simulation 3D geometry of the enclosure of FIG. 29 with a supine patient exhaling particles at an average breathing rate of 10 L/min;

FIG. 37B is a mesh of the simulation of FIG. 37A with 800,00 cells;

FIG. 37C is a graph of representative results of a simulation for 0.1 micron particle size emissions;

FIG. 38 is a rear perspective view of an enclosure according to still another exemplary embodiment of the present invention;

FIG. 39 is an exploded perspective view of the panel portions of the enclosure of FIG. 38;

FIG. 40 is an exploded perspective view of the panel portions of the enclosure of FIG. 38, showing flexible material openings;

FIG. 41 is a front elevational view of the enclosure of FIG. 38;

FIG. 42 is a rear elevational view of the enclosure of FIG. 38;

FIG. 43 is a side elevational view of a septum for passing items into the enclosure of FIG. 38;

FIG. 44 is a perspective view of a port used with the enclosure of FIG. 38 with a suction tube extending through the port; and

FIG. 45 is a perspective view of the enclosure of FIG. 38 in use with a patient.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term “interior” means that part of the inventive enclosure within or between the panels and/or flexible sheet and “exterior” means all else not within or between the panels and/or flexible sheet.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/- 10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

The present invention provides protection for medical personnel when treating a patient, particularly during an intubation procedure.

When patients in respiratory distress are intubated and extubated, bodily fluids or aerosols can spray into the surrounding air, causing risk of transmission of infectious diseases. The current invention provides an enclosure device that can be placed over a patient in order to contain emissions, as well as any aerosols containing micro-and nano-sized particles. The device can be collapsible and can be stored in a collapsed, flat condition. The device can be constructed from plastic (e.g., polycarbonate) sides with clear plastic sheet (e.g. vinyl) as the back and top panels. The sides and back of the device contain access ports for physicians or their instruments to enter the patient’s area. These ports may be holes, vertical slits, or similar access ports that overlap so that they form a sufficient seal while also allowing easy access to the patient when pushed aside. There is the possibility of including filters or negative air flow ports. The entire device, or just portions of the device, can be disposable. The device may also have straps that can be tightened underneath a bed, a gurney, or a stretcher on which a patient is lying to secure the entire device in place. The device can be disinfected after use, such as by wiping down or spraying with a disinfectant, autoclaving, or other known disinfecting process or method.

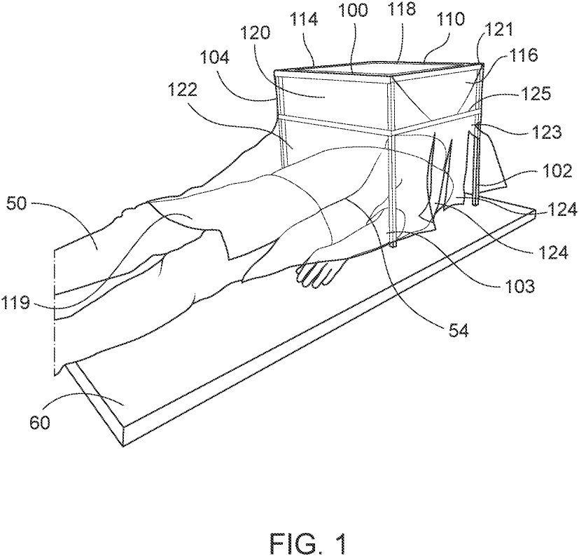

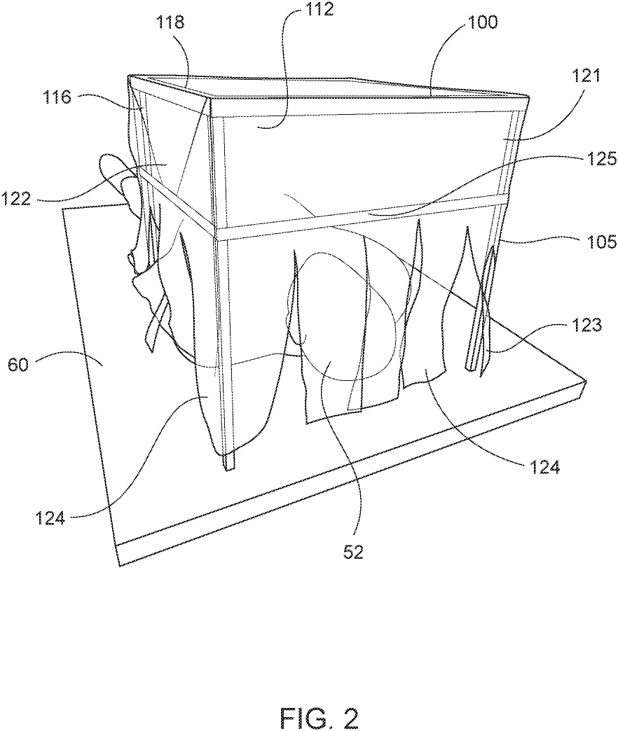

Referring to FIGS. 1-5, the protection is provided by an intubation protection enclosure (“enclosure 100”) that covers a head 52 and torso 54 of a patient 50 when the patient 50 is lying on his/her back. Enclosure 100 includes a frame 110 sized to extend over the head 52 and torso 54 of an adult human patient 50 and a transparent (or translucent) material 122 attached to frame 110 to allow medical personnel to view patient 50 when patient 50 is placed under enclosure 100. Material 122 can be flexible or rigid, according to a desired configuration.

Frame 110 can be constructed from polymer tubing, aluminum, or other lightweight, structurally sturdy material. Frame 110 can be of unitary construction or can be separate pieces that are fit together at or near the point of use. Further, frame 110 can be designed to pop open like a tent or an umbrella. Frame 110 generally defines a cube or parallelepiped structure with four corner legs 102, 103, 104, 105. Front and rear securing straps 106, 108 are attached to legs 104, 105, respectively, and are releasably connectable underneath a stretcher 60, a gurney, a table or other horizontal patient support to legs 103, 102, respectively, to secure enclosure 100 to stretcher 60, the gurney, the table or other horizontal patient support.

Frame 110 also includes a rear portion 112, a left portion 114 attached to rear portion 112, a right portion 116 attached to rear portion 112, distal from left portion 114, and a top portion 118 attached to rear portion 112, left portion 114, and right portion 116. A front portion 120, distal from rear portion 112, is attached to top portion 118, left portion 114, and right portion 116.

Frame 110 has an upper perimeter 121 and a lower perimeter 123, defined by a horizontal rib 125 extending around the full perimeter of frame 110. Optionally, one side of the horizontal rib 125 may be removed to increase the field of view. In an exemplary embodiment, legs 102, 103, 104, 105 have an overall length of between about 18 inches and about 30 inches, while upper perimeter 121 has a height of between about 8 and about 16 inches.

A transparent flexible sheet 122 is attached to the upper perimeter 121 of the frame 110. The flexible sheet 122 can be a single unitary sheet of transparent/translucent vinyl or other non-permeable transparent/translucent material. Alternatively, flexible sheet 122 can be constructed from a plurality of sheets that are sewn or otherwise joined together. Flexible sheet 122 covering upper perimeter 121 is preferably transparent to allow medical personnel to view into enclosure 100 to observe patient 50.

Sheet 122 is placed over at least part of frame 110 such that sheet 122 covers top portion 118, left portion 114, right portion 116, rear portion 112, and front portion 120. Gravity is used to weight sheet 122 downwardly. Optionally, weights can be provided in sheet 122 to help maintain sheet 122 over frame 110. Sheet 122 also extends at least partially around the lower perimeter 123 between legs 104 and 105, between legs 105 and 102, and between legs 102 and 103. In an exemplary embodiment, sheet 122 comprises a plurality of vertical strips 124 along left portion 114, right portion 116, and rear portion 118 to allow medical personnel to reach into enclosure 100 to work on patient 50. In order to minimize the flow of air or particulate from inside enclosure 100, vertical strips 124 comprise a first row of strips 126 and a second row of strips 128 behind first row of strips 126 and offset from first row of strips 126 to form a wall of strips 124 such that each strip 124 in one of rows 126, 128 overlaps adjacent strips 124 in the other row 128, 126. When medical personnel are not working on patient 50, strips 126, 128 form a sufficient seal to prevent bodily fluids, sprays, aerosols, or other airborne particulates from escaping to outside enclosure 100.

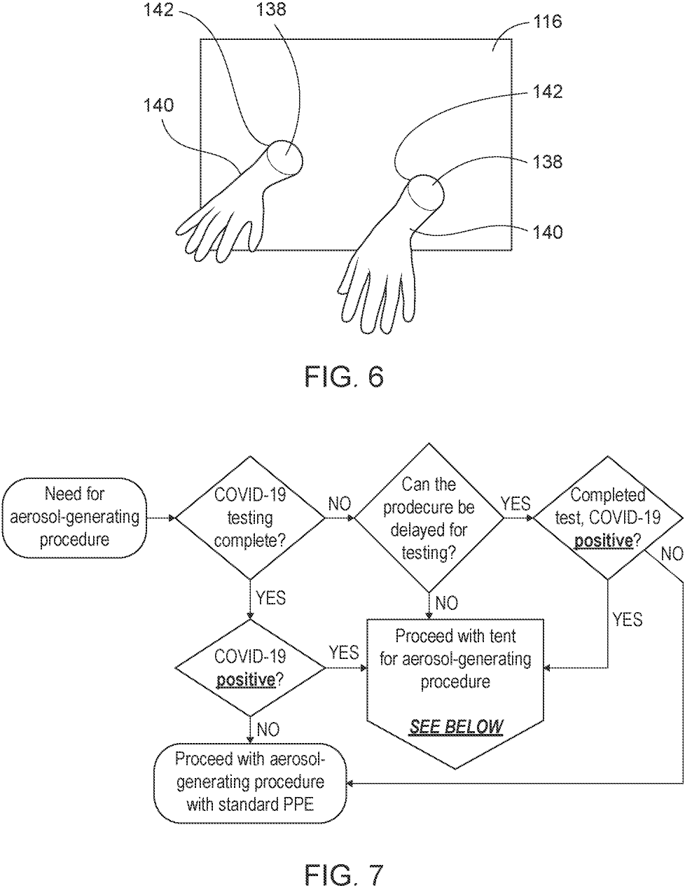

Referring to FIG. 5, sheet 122 can be formed from a single planar sheet. Sheet includes a generally rectangular top portion 118 with left and right opposing wings forming left and right portions 124, 116, respectively. Rear portion 112 extends from a side of top portion between left and right portion 114, 116. Strips 124 can extend from rear portion 112 such that strips 124 will extend below rib 125. While strips 124 are only shown in FIG. 6 with respect to rear portion 112, those skilled in the art will recognize that strips 124 can be formed in left and right portions 114, 116, as shown in FIGS. 1-4. A flap 119 extends between eft and right portions 114, 116 away from rear portion 112 and covers a lower body portion of patient 50, as shown in FIG. 1.

Referring to the double headed arrows A, B, C, D in FIG. 5, the associated sides of sheet 122 are attached to each other to form enclosure 100. The sides can be ultrasonically welded to form airtight bonds. Alternatively, when sheet 122 is placed over frame 110, the sides can be taped or otherwise secured to each other, as desired.

Rear portion 112 has a through-opening 138 formed therethrough, with a cover 140 over through-opening 138. Through-opening 138 is sized to allow access through sheet 122 to allow the insertion of instrumentation, tubes, or other equipment (not shown) to pass between the interior and the exterior of enclosure 100.

Additionally, as shown in FIG. 6, right portion 116 can include through-openings 138 with gloves 140 such that each glove 140 has a glove opening 142 defining through-opening 138. Glove 140 extends into the interior of enclosure 100. Additional openings can be provided in any of top portion 118, left portion 114, right portion 116, and rear portion 112 to allow the insertion of instrumentation, tubes, or other equipment to pass between the interior and the exterior of enclosure 100 or to draw air from inside enclosure 100.

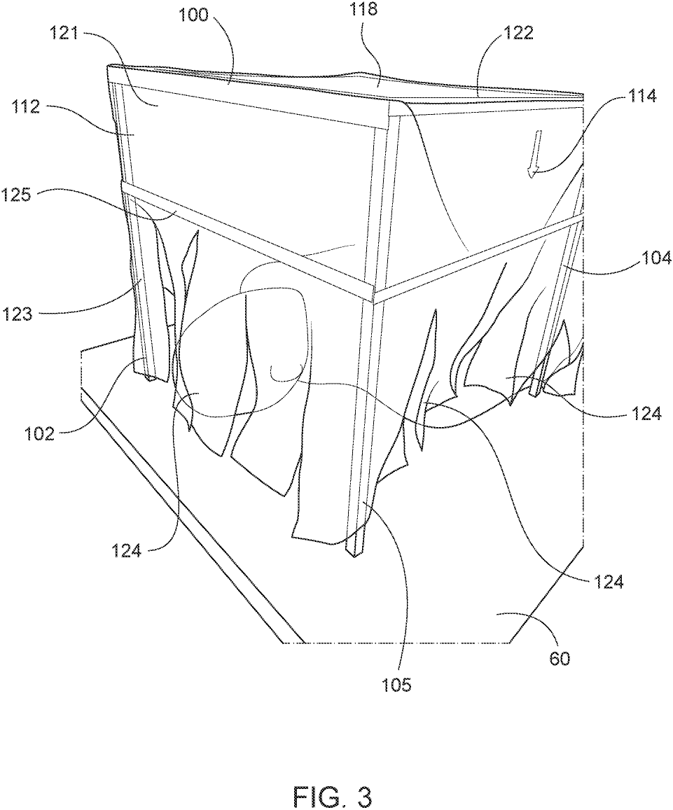

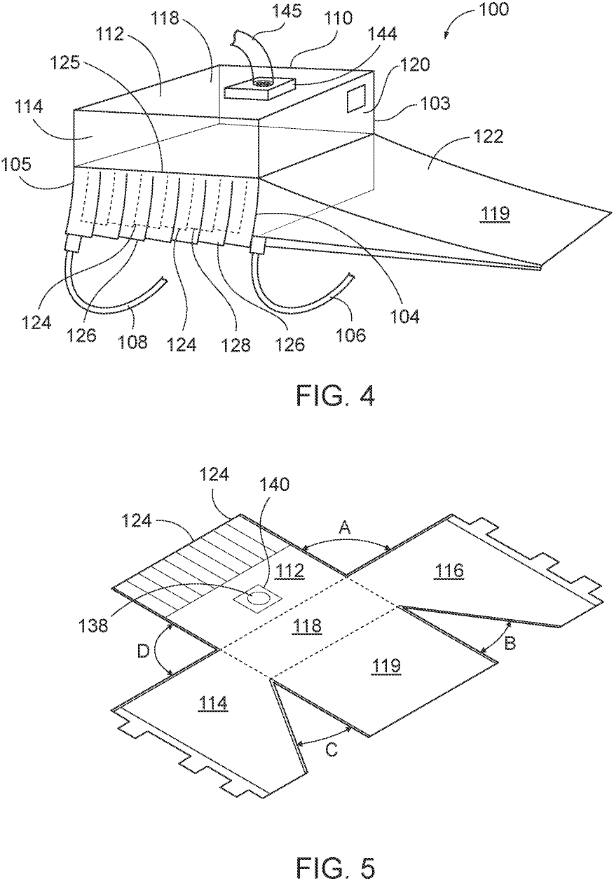

Optionally, as shown in FIG. 4, a fan 144 can be attached to one of top portion 118, left portion 114, right portion 116, and rear portion 112 so that fan 144 can draw air from inside enclosure 100. While fan 144 can be directly attached to sheet 122, those skilled in the art will recognize that fan 144 can be connected to a through-opening in sheet 122, with ducting or other tubing 146 (shown in FIG. 11) between fan 144 and sheet 122. Discharge from fan 144 can be directed away from enclosure 100 via ducting 145.

Optionally, a bottom panel (not shown) can be provided under frame 110. If used, the bottom panel can either be connected to frame at the bottoms of legs 102, 103, 104, 105 or, alternatively, the bottom panel can be separate from frame 110 and slid under head 52 of patient 50 prior to or after placing frame 110 around patient 50.

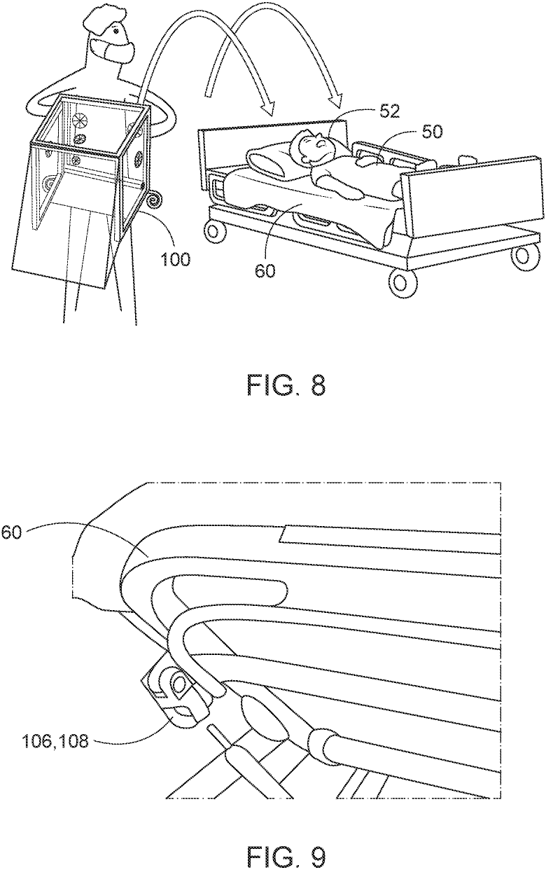

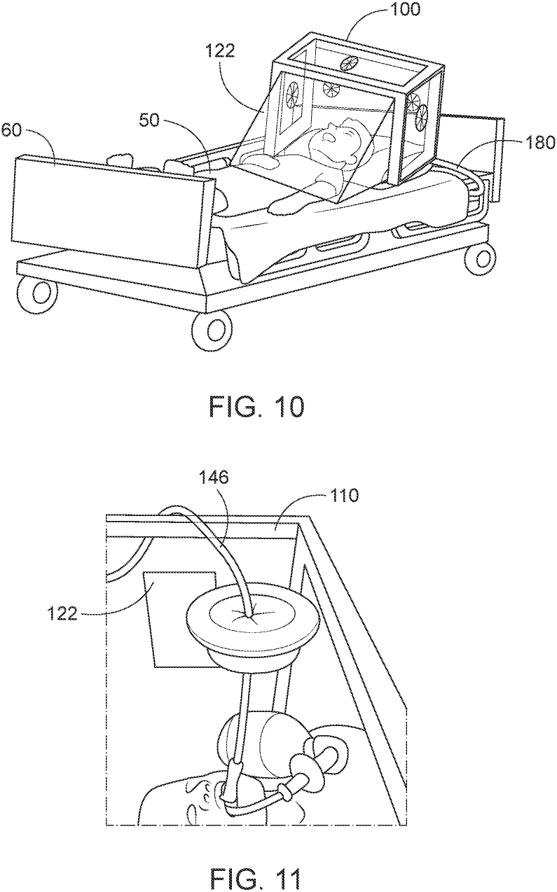

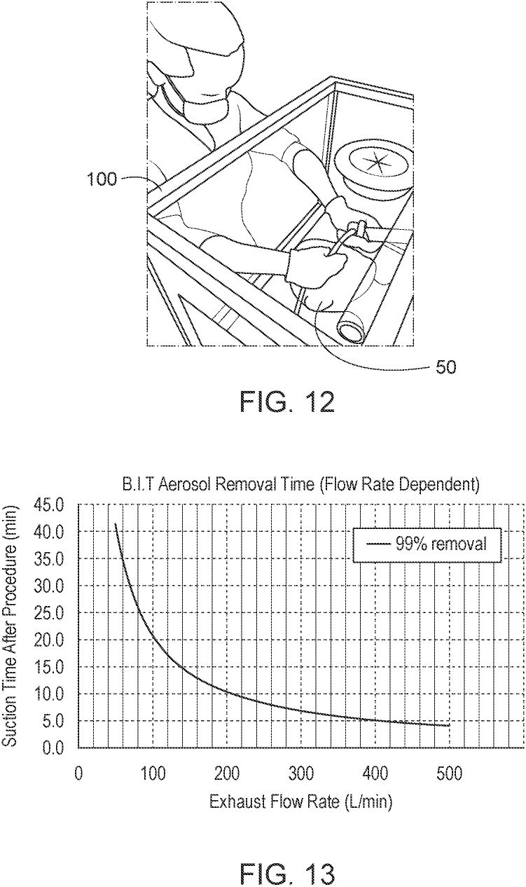

FIGS. 7-12 illustrate exemplary steps to set up and use enclosure 100 over a patient 50. FIG. 7 provides an exemplary flowchart to determine whether enclosure 100 is needed for a particular patient 50. FIG. 8 illustrates the step of placing enclosure 100 over head 52 of patient 50. FIG. 9 illustrates the step of securing straps 106, 108 underneath the bed 56 on which patient 50 is lying. FIG. 10 illustrates the step of tucking in sides of sheet 122 under patient 50. FIG. 11 illustrates the step of connecting suction tubing 146 to sheet 122. FIG. 12 illustrates the step of a clinician 90 working on patient 50 through enclosure 100.

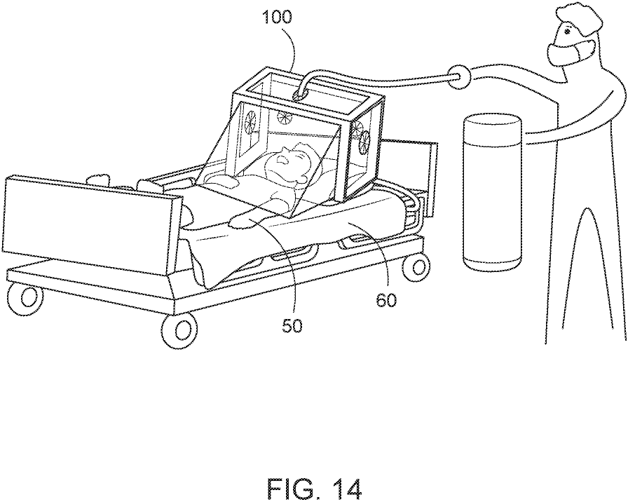

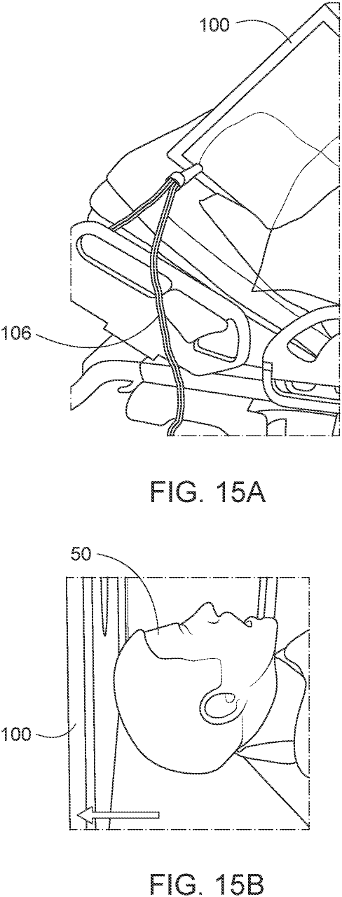

FIG. 13-15B illustrate exemplary steps to begin decontamination of enclosure 100 while still in place over patient 50, remove enclosure 100 from patient 50, clean the interior and exterior of enclosure 100, inspect enclosure 100 for defects, cuts or tears, and store enclosure 100 for later use. Specifically, FIG. 13 shows an exemplary graph of the amount of time required to draw air from inside enclosure 100 to achieve approximately 99% removal of potentially contaminated air from inside enclosure 100 as a function of the exhaust air flow rate. FIG. 14 illustrates the removal of tubing from enclosure 100. FIGS. 15A-15B illustrate the removal of straps 106, 108 and removal of enclosure 100 from patient 50. After enclosure 100 is removed, interior of enclosure 100 is decontaminated. Then, exterior of enclosure 100 is decontaminated and enclosure 100 is stored for future use.

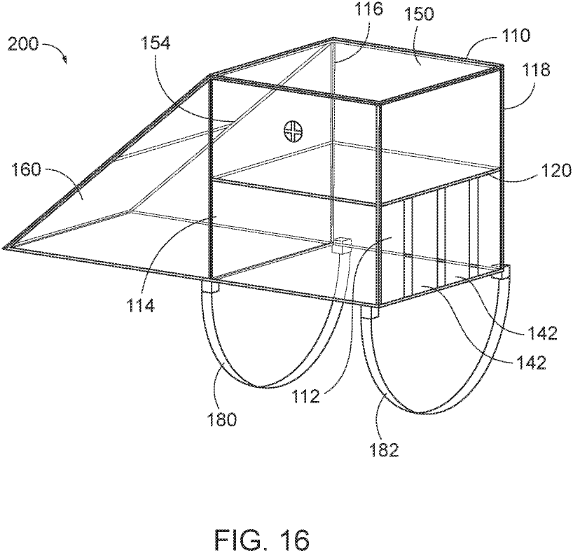

FIG. 16 shows an alternative exemplary embodiment of an enclosure 200 according to the present invention. Enclosure 200 is similar to enclosure 100, but also includes additional members of frame 110. Specifically, sloped frame members 190, 192 extend along the length of flap 160, with central and bottom frame pieces 193, 194, respectively, connecting sloped frame members 190, 192.

Additionally, lower frame pieces 195, 196, 197 join bottom frame piece 194 to form a full lower frame perimeter of enclosure 200, with a brace member 198 extending between the bottom of leg 114 and the bottom of leg 116.

While a flexible sheet 122 is used in the present embodiment, those skilled in the art will recognize that portions of enclosure 100 or enclosure 200 can be covered in a rigid transparent material, such as plexiglass. By way of example only, plexiglass can be used for the top and upper perimeter 120 of enclosure 100 or enclosure 200 and can be used to enhance the structural integrity of enclosure 100 or enclosure 200.

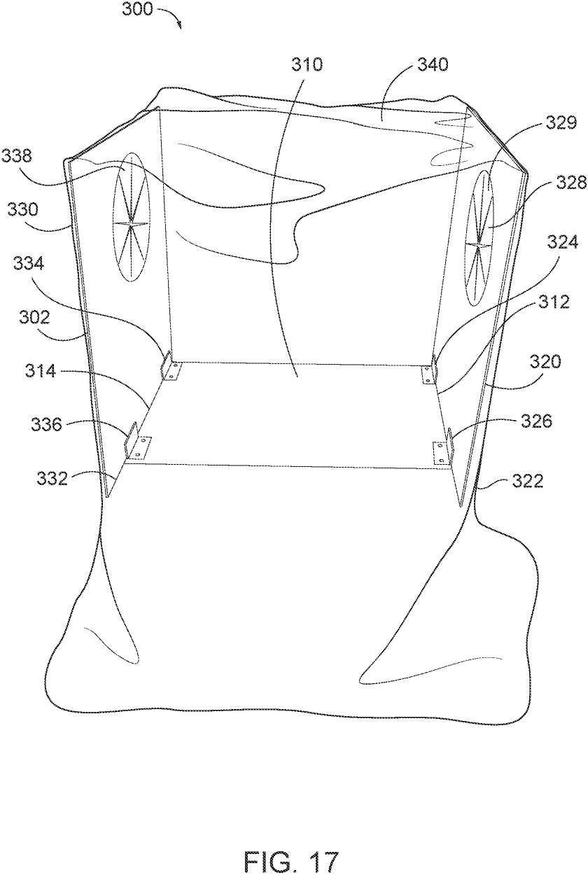

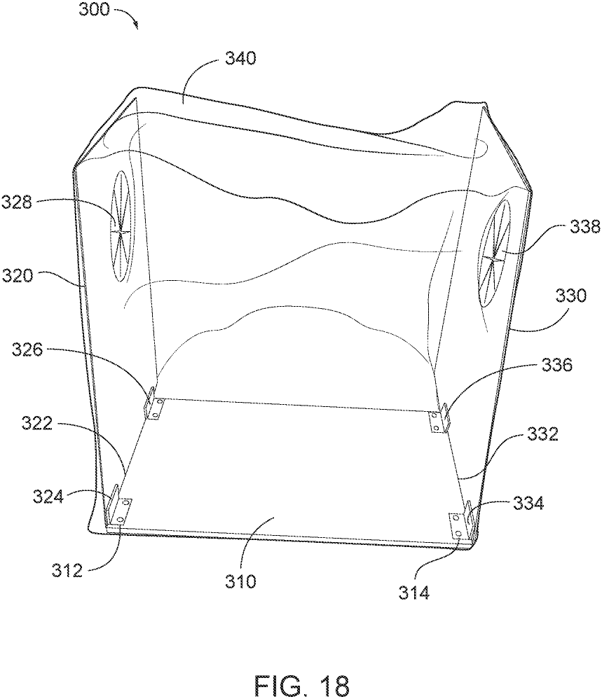

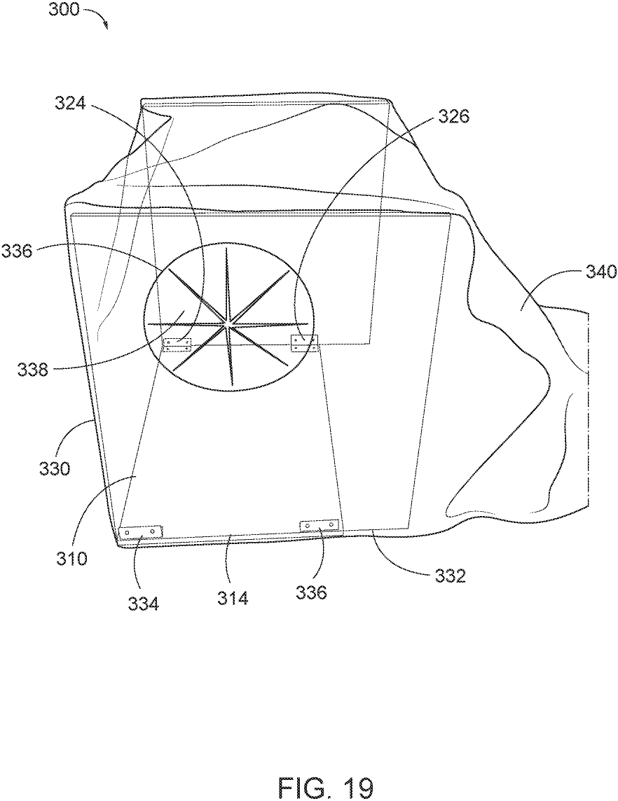

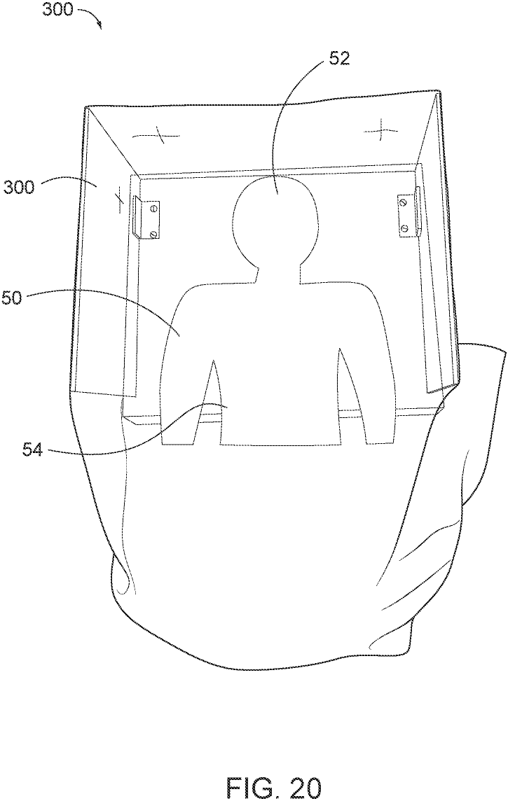

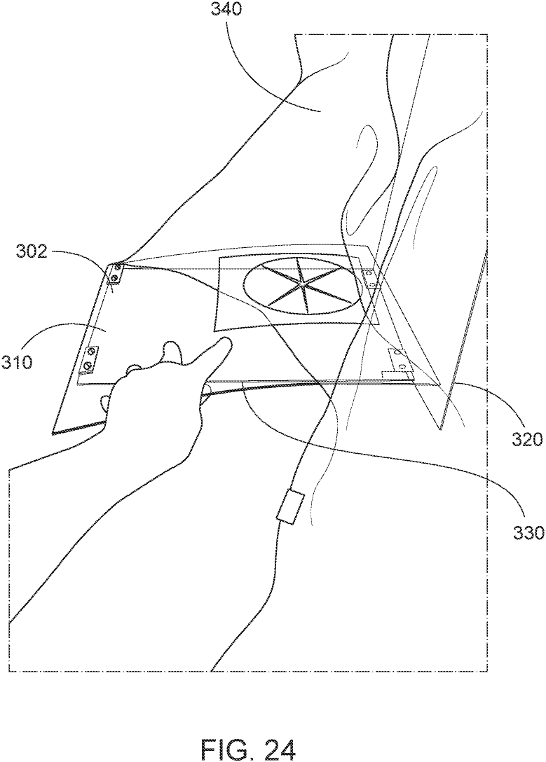

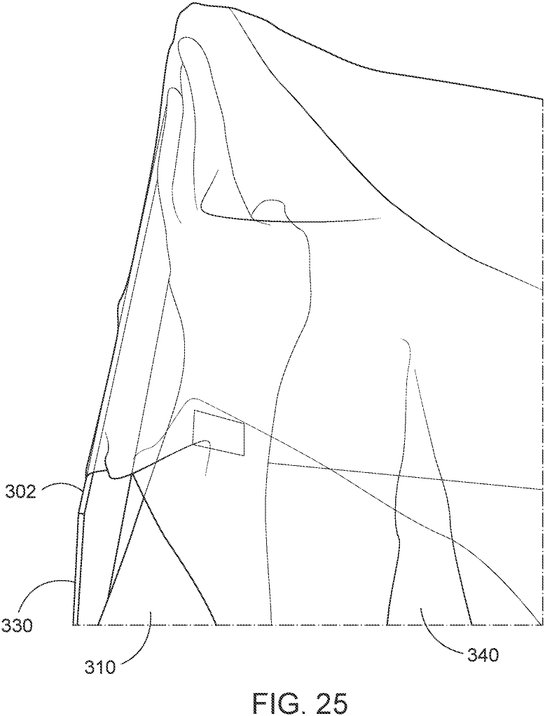

An alternative exemplary embodiment of an enclosure 300 according to the present invention is shown in FIGS. 17-28. Enclosure 300 includes a frame 302 having a base 310 constructed from a rigid material. Base 310 includes a right side 312 and a left side 314, each connected to base 310 and foldable toward base 310 to collapse enclosure 300 when not in use.

A right side wall 320 has a lower right side 322 hingedly connected to right side 312 via hinges 324, 326. A generally circular right side opening 328 is formed in right side wall 320. Right side opening 328 is sized to allow a clinician to insert his/her hand (not shown) through right side wall 320 to access the patient inside enclosure 300.

Similarly, a left side wall 330 has a lower left side 332 hingedly connected to left side 314 via hinges 334, 336. A generally circular left side opening 338 is formed in left side wall 330. Left side opening 338 is sized to allow a clinician to insert his/her hand (not shown) through left side wall 330 to access the patient inside enclosure 300. Side openings 328, 338 can be covered with a flexible member 329, 339, respectively, having a plurality of slits emanating from a center of member 329, 339 to close side openings 328, 338 when a clinician’s hand is not extended through either side opening 328, 338.

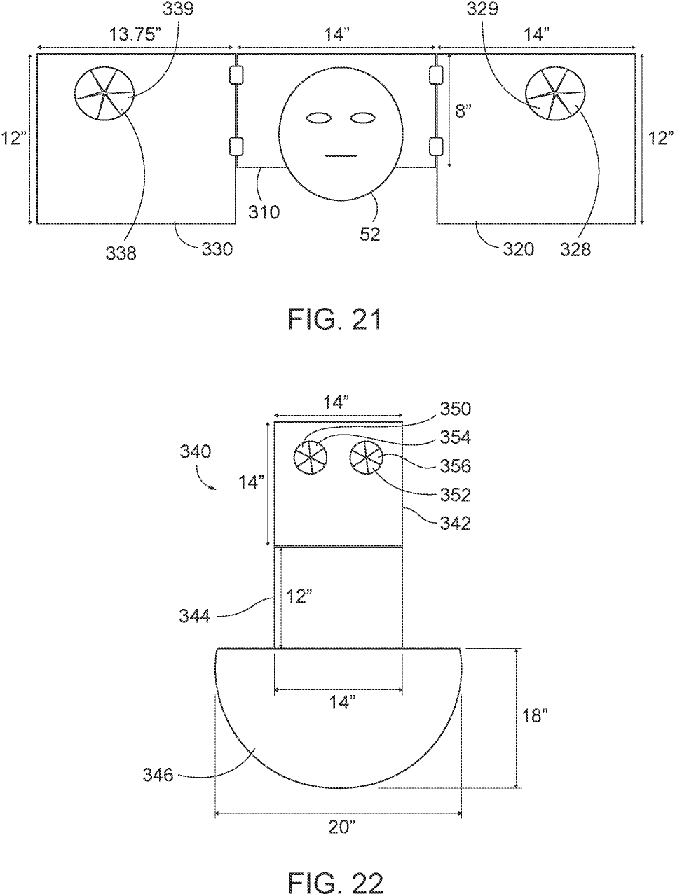

Right and left side walls 320, 330, respectively, can be constructed from a transparent or translucent material, such as polycarbonate, acrylic, or other rigid material. As shown in FIG. 21, right side 320 and left side 330 can extend beyond the width of base 310. While exemplary dimensions of base 310, right side 320, and left side 330 are shown in FIG. 21, those skilled in the art will recognize that frame 302 can be constructed from components having different dimensions.

A flexible drape 340, shown in FIG. 22, is connected to frame 310 and forms a rear portion 342, top portion 344 (forming a roof), and front portion 346 of enclosure 300. Drape 340 can be constructed from a transparent or translucent material, such as polyethylene-vinyl acetate (PWVA), polyvinyl chloride (PVC) or other suitable material.

Rear portion 342, as shown in FIG. 22, can include a plurality of rear openings 350, 352 that can be hand and/or instrument portions to allow access to the patient from outside of enclosure. Similar to side openings 328, 338, rear openings 350, 352 can be covered with a flexible member 354, 356, respectively, having a plurality of slits emanating from a center of member 354, 356 to close rear openings 350, 352 when a clinician’s hand or instrumentation is not extended through either rear opening 350, 352. In an exemplary embodiment, rear portion 342 can be a 14″ x 14″ square.

Top portion 344 extends between right and left side walls 320, 330, respectively, and from rear portion 342 toward a front of frame 302. Top portion 344 can be a solid sheet or, alternatively, through openings (not shown) can be provided for instrumentation access. In an exemplary embodiment, top portion can be 14″ x 12″.

A forward portion 346 extends from top portion 344, distal from rear portion 342, and is used to cover the front of frame 302, as well as at least a portion of the patient’s torso 54. Forward portion 346 can be semi-circular in shape, as shown in FIG. 22. Alternatively, forward portion 346 can be other shapes.

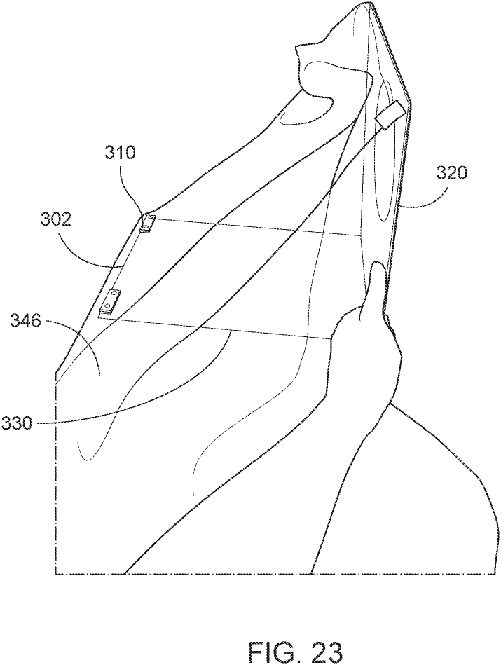

Referring to FIGS. 23-28, to assemble enclosure 300, start with enclosure 300 orientated such that base 310 is on the underside and the back of the side walls 320, 330 are facing the builder. Unfold the flexible material 340. Referring to FIGS. 23-25 specifically, grasp the bottom edge of the top-most side wall 320, 330 and torque outward. Grasp the outer edge of the remaining side wall 330, 320 and torque outward. The builder uses his/her hand to guide the corner of flexible drape 340 into place.

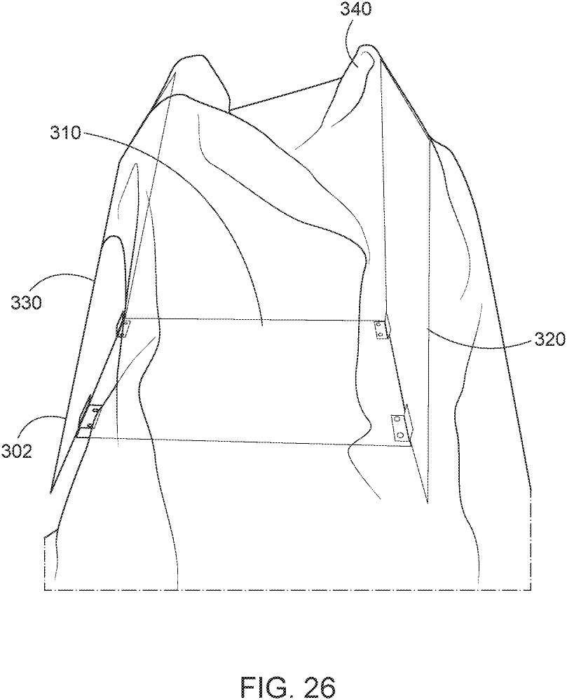

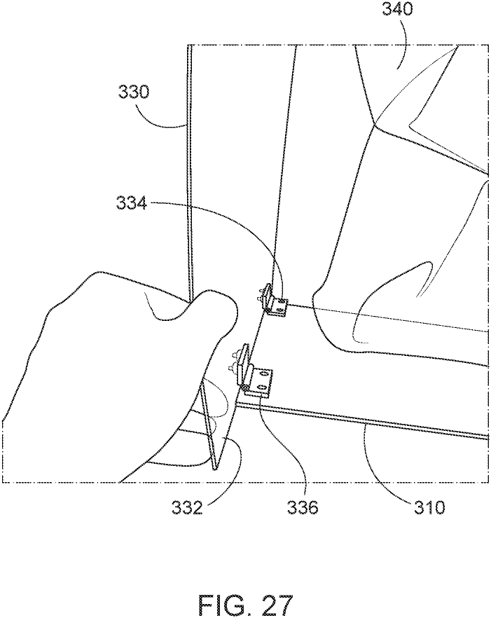

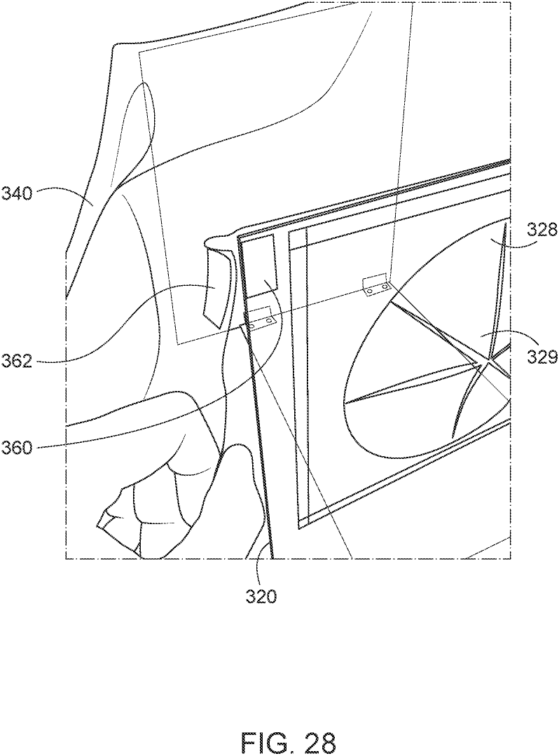

Enclosure 300 will look as shown in FIG. 26. Using both hands, grasp the bottom edges of both side panels and torque outward forcefully, attempting to torque past 90 degrees, as shown in FIG. 27. This enables frame 302 to hold tension across flexible drape 340. Slide the grey panel under the patient’s head 52 and secure drape 340 to the device 302 using a releasable connection, such as hook and loop tabs 360, 362 on both side panels 320, 330, as shown in FIG. 28. Tuck excess flexible material 340 around the patient’s collarbone area to minimize gaps around flexible material 340.

When using enclosure 300, when accessing enclosure 300 from either side wall 320, 330, the user should ensure that the interior port flap 329, 339 lays flat when hands are retracted to maintain an adequate barrier.

To disassemble enclosure 300 after use, remove drape 340 from the side walls 320, 330. Starting with side wall 330, as side wall 330 is being collapsed downward toward base 310, guide the flexible material 340 away from the rear corner of side wall 330. Repeat with side wall 320 and, after side wall 320 is collapsed onto side wall 330, wrap excess flexible material 340 around frame 302.

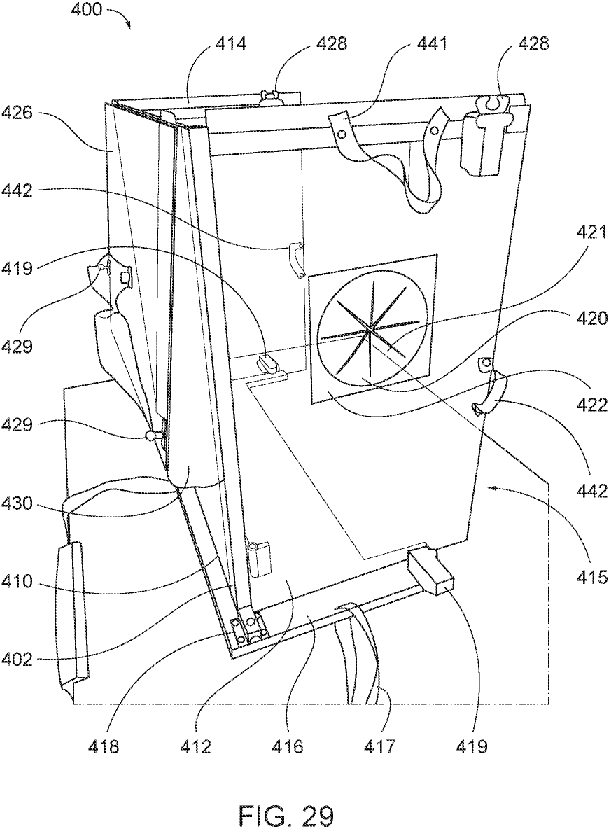

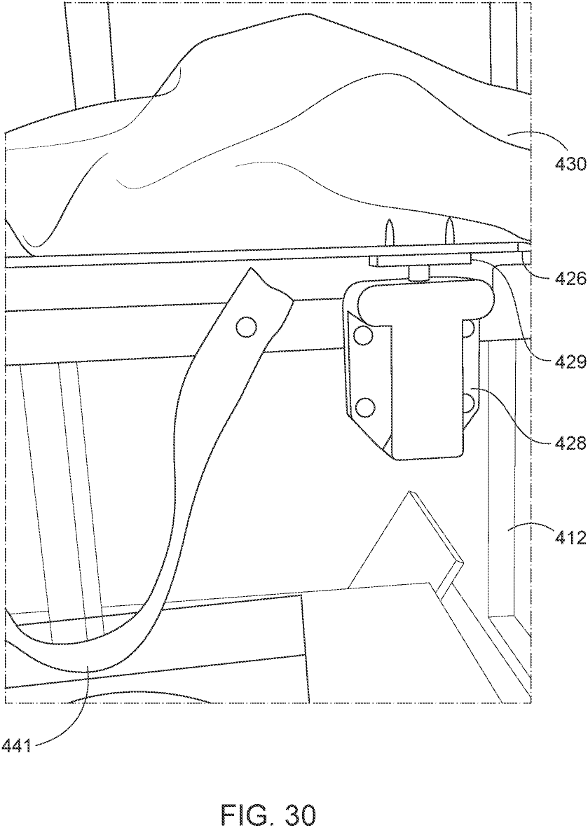

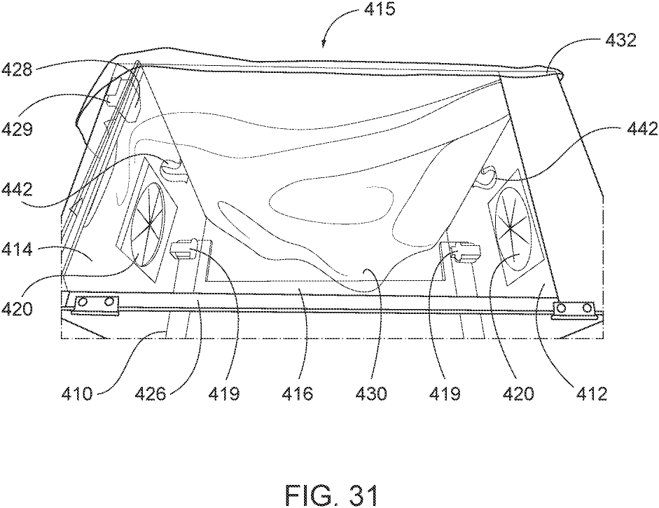

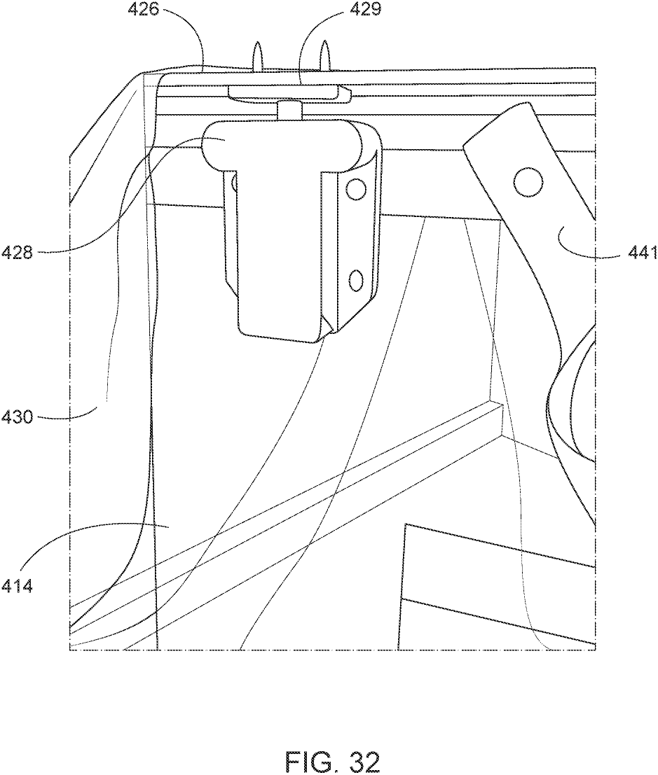

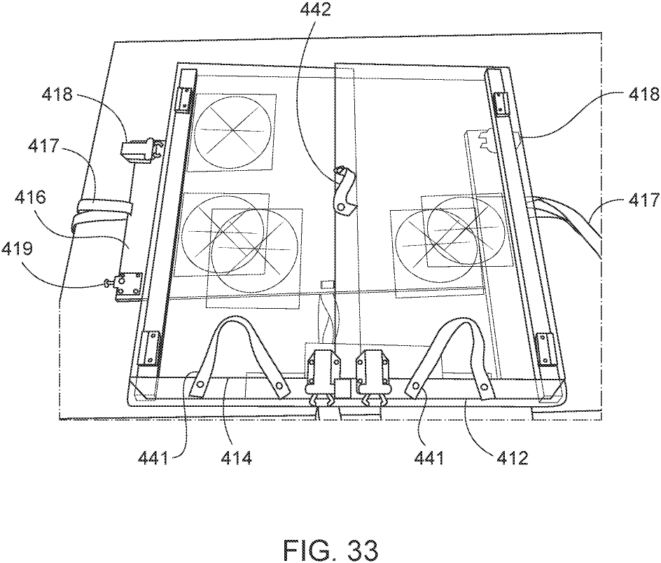

An alternative exemplary embodiment of an enclosure 400 according to the present invention is shown in FIGS. 29-35. Enclosure 400 includes a frame 402 having a rear panel 410, a left panel 412, and a right panel 414 such that each of left panel 412 and right panel 414 is hingedly connected to rear panel 410. An open front 415 is formed between side panels 412, 414, distal from rear panel 410. A base panel 416 is releasably attached to left panel 412 and right panel 414 via rear clamps 418 and side clamps 419. In an exemplary embodiment, clamps 418, 410 can be Window Cabinet Button Spring Loaded Automatic Door Latch Lock Furniture Cabinet Drawer Door Bolt with Spring Button, manufactured by WY.

Rear panel 410 has a length at least as long as the total sum of the length of left panel 412 and the length of right panel 414 so that, when left panel 412 and right panel 414 are folded toward base 416 for storage, each of left panel 412 and right panel 414 can lay flat on top of base 416, reducing the overall height of frame 402 in the folded position.

Left panel 412, right panel 414, and rear panel 410 each comprises a rigid transparent panel. As shown in FIG. 29, at least one panel (in FIG. 29, left side panel 412) has a through-opening 420 extending therethrough and is sized to allow the introduction of a clinician’s hand and forearm (not shown). Through opening 420 can be 5″ in diameter, although those skilled in the art will recognize that through-opening 420 can be other sizes as well. A flexible cover 421 is removably placed over through-opening 420 to seal through-opening 420 when not in use. In an exemplary embodiment, flexible cover 421 can be a plurality of pie-shaped pieces, with a solid flexible plastic flap 422 attached to the interior of the panel to seal through-opening 420 when not is use.

As discussed above with respect to enclosure 100, one or more gloves (such as glove 140 discussed above) can be attached to at least one of panels 410, 412, 414 at through-opening 420 to allow a clinician (not shown) to insert his/her hand and forearm into the glove to reach inside enclosure 400.

A roof 426 is hingedly attached to the top of rear panel 410 and can be flipped onto the tops of side panels 412, 414. Spring loaded latches 428 can be affixed toward the front portions of each of side panels 412, 414, with a complementary insert 429 attached to roof 426. An exemplary latch/insert can be a Non-Magnetic Push to Open Catch Lock Drawer Cabinet Catch Touch Latch manufactured by Light up in the Dark.

Sheet 430 can be transparent, translucent, or opaque and can be a plastic sheet or a woven fabric. Optionally, smart sensors can be embedded in the fabric. Such sensors can measure CO₂, relative humidity (“RH”), particulate matter, or other useful parameters. These sensed parameters can be used to indicate when the enclosure is sufficiently evacuated or provide other information about the patient or enclosure condition. These parameters are indirect measures for exhalation or aerosol release from patient 50 and whether the concentrations of exhale gases and/or aerosols have been reduced, such as via an attached vacuum line.

In an exemplary embodiment, sheet 430 is clamped to panels 410, 412, 414 with clips, such as removable binder clips or similar type clips that can be readily removed in the event that patient 50 requires direct intervention. Alternatively, two-sided tape 432 can be pre-applied to the edges of panels 410, 412, 414 and sheet 430 can be affixed to the tape so that sheet 430 is draped over front 415 to cover patient 50. Still alternatively, full-length clips can be provided along the front edges of panels 412, 414 and roof 426with mating inserts attached to sheet 430 so that the inserts can be releasably inserted into the clips.

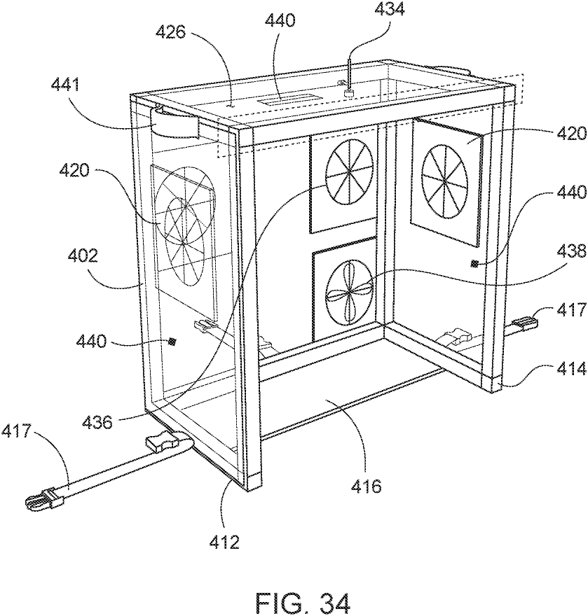

Referring to FIG. 34, in addition to hand through-opening 420, other through-openings 434 can be provided in panels 410, 412, 414, and roof 426. Such through-openings 434 can be used to allow instrumentation, intubation tubes, vacuum tubes, or other devices (not shown) to pass through and into enclosure 400. To help maintain a seal around such devices, through-openings 434 can be provided with elastomer seals having a central opening to allow the device to pass.

Further, a lateral slit 440 is provided in roof 426 to allow for the insertion of larger devices, such as a camera or other medical device (not shown) into enclosure 400. Similar to through-openings 434, an elastomer seal with a central slit is provided to help maintain a seal around such devices.

Additionally, a vent/fan/vacuum pump opening 436 can be provided in any one of panels 410, 412, 414, base 416, or roof 426. Opening 436 can have attached ducting (nor shown) for connection to a vent fan and can be configured for connection to a hospital vacuum line. Optionally, a fan 438 can be fixed mounted on one of panels 410, 412, 414 or roof 426 to aid in evacuation aerosols from inside of enclosure 400.

Further, while sensors have been disclosed as incorporated into sheet 430, alternatively, sensors 440 can be mounted on the insides of panels 410, 412, 414, and/or roof 426. Such sensors 440 can measure CO₂, RH, particulate matter, or other important parameters.

Optionally, handles 441, 442 can be provided on the exterior of side panels 412, 414 to assist in carrying enclosure 400 as well as opening/closing enclosure 400. As seen in FIG. 29, handles 441 can be located toward the top of side panels 412, 414 and handles 442 can be located toward the front of side panels 412, 414.

Enclosure 400 can be disposable. Alternatively, enclosure 400 can be re-used after disinfecting according to exemplary procedures discussed above. The materials from which enclosure 400 is constructed are to be impervious to standard hospital cleaning agents. Portions or all of enclosure 400 can be autoclaved, whereas sheet 430 can be laundered. Alternatively, sheet 430 can be disposable.

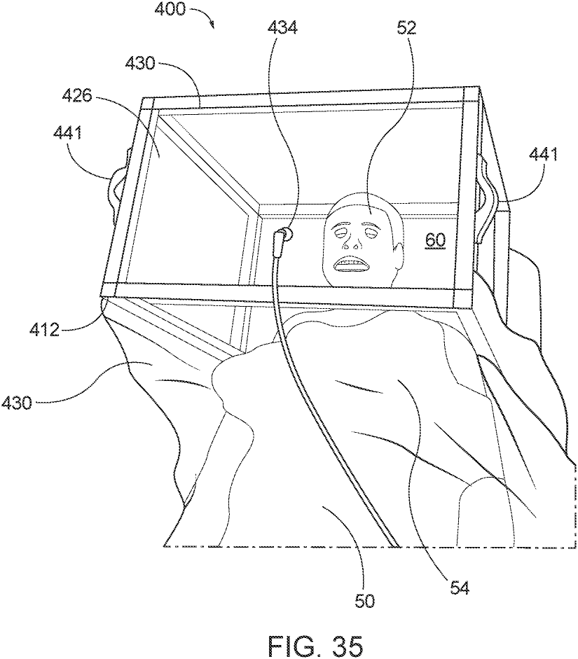

To assemble enclosure 400, base 416 is laid on a bed or stretcher 60 such that rear clamps 418 are positioned beyond head 52 of patient 50 and side clamps 419 are adjacent head 52 of patient 50, toward the patient’s shoulders. Optionally, securing straps 417 can be secured to base 416 and can be secured underneath the bed or stretcher 60 to secure base 416 to the bed or stretcher 60. The straps 417 are sufficiently long to allow for securing of enclosure 400 when the patient 50 is supine, upright, or other typical positions. Straps 417 include quick release latches to allow for rapid installation/removal to/from a bed or stretcher 60. By way of example only, FIG. 35 shows a patient 50 in a partially upright position, between supine and totally upright. If base 416 is omitted, straps 417 can be attached to each of side panels 412, 414.

Patient 50 can be placed on base 416. Frame 402 is then placed over top of base 416 and left and right side panels 412, 414, respectively, are opened about 90 degrees with respect to rear panel 410. Frame 402 is lowered onto base 416. Rear panel 410 is snapped into rear clamps 418 and left and right panels 412, 414 are further opened a full 90 degrees with respect to rear panel 410 to snap into side clamps 419. Roof 426 is rotated over top of side panels 412, 414 so that clamp insert 429 is inserted into clamp 428 to secure the front of roof 426 to side panels 412, 414. Flexible sheet 430 can then be draped over the front of enclosure 400.

To disassemble enclosure 400, patient 50 is removed from enclosure 400. Side clamps 419 are unlocked and folded inward toward each other, releasing left and right panels 412, 414 from base 416. Rear clamps 418 are next unlocked, allowing frame 402 to be lifted from base 416. Left and right panels 412, 414 are folded fully toward rear panel 410, collapsing sheet 424 inwardly into frame 402. Straps 417 are undone. Frame 402 and base 416 can then be removed and disinfected and/or stored for future use.

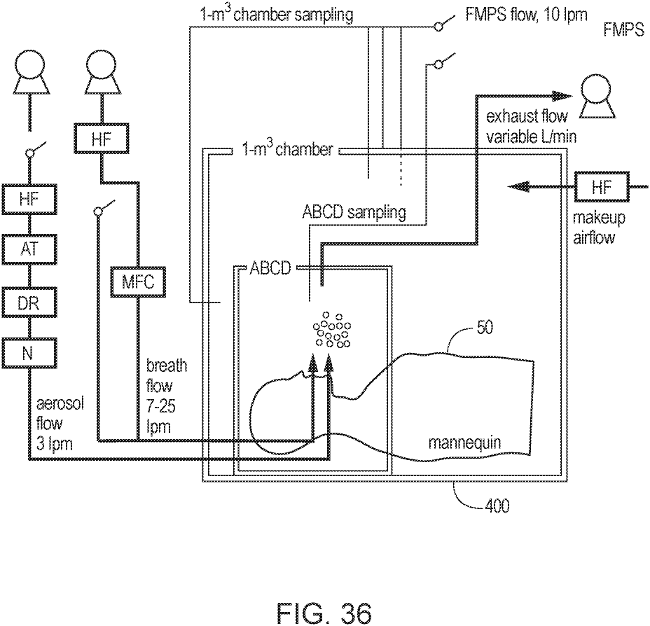

FIG. 36 is a schematic drawing of a 1 m³ stainless steel chamber system for aerosol evaluation testing. HF is a HEPA filter; AT is an atomizer; DR is a diffusion drier; N is a neutralizer; MFC is a mass flow controller; FMPS is a TSI Fast Mobility Particle Sizer 3091, which measures particles in a range of 0.0056 microns to 0.56 microns in a size-resolved basis over 32 bins at a time-resolution of 1 second.

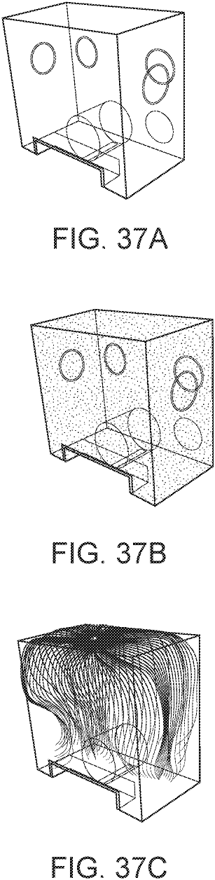

FIG. 37A shows a simulated geometry of the enclosure 400 with a patient in a supine position exhaling particles at an average breathing rate of 10 L/min. FIG. 37B shows a mesh of the enclosure of FIG. 37A with 800,000 cells. FIG. 37C shows representative results of a simulation for 0.1 micron particle size emissions where darker particles in the bottom/middle indicate a short duration (age of the particle) after emission and the lighter particles at the top/sides indicate long duration, with some particles trapped in the corners of the enclosure.

An alternative exemplary embodiment of an enclosure 500 according to the present invention is shown in FIGS. 38-45.

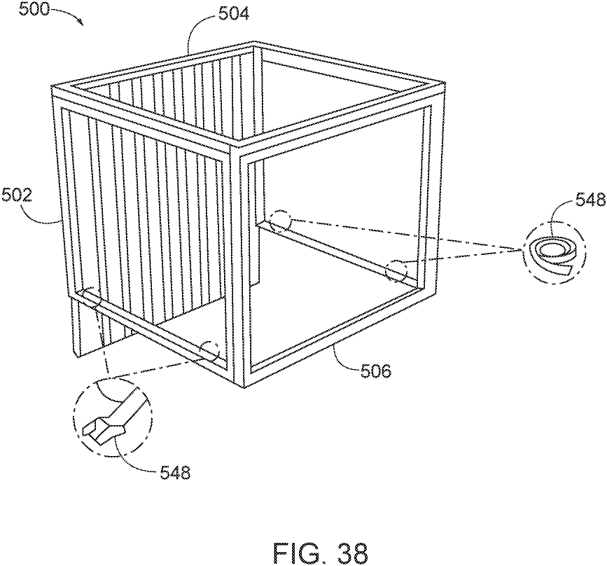

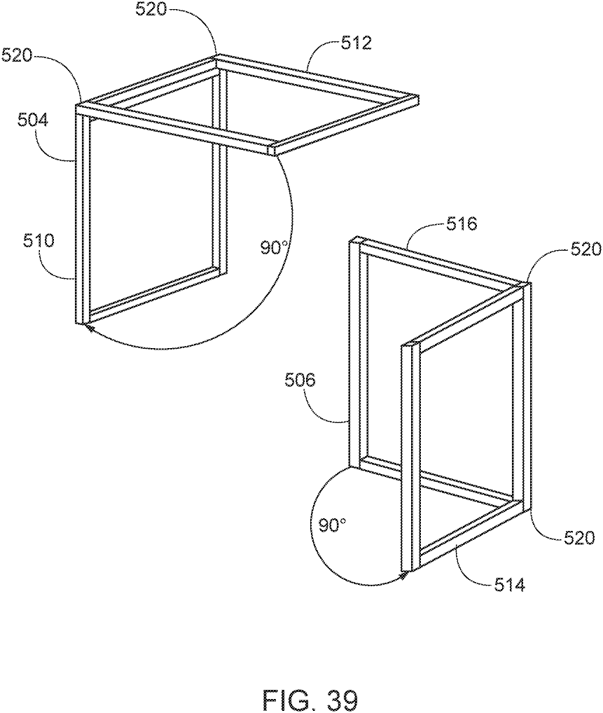

Enclosure 500 can include a frame 502 that is separable into multiple parts, for example, a first part 504 and a second part 506 as shown in FIG. 39. The frame 502 may be formed of an aluminum or other relatively sturdy material. Each of the parts 504, 506 may be transitionable from an expanded condition in which part 504 and part 506 each form two sides of a cube to a collapsed condition in which sides 510, 512 of part 504 and sides 514, 516 of part 506 lie flush against one another. In this manner, the frame 502 may be easily stored when not in use. The frame 502 may include hinges 520 as shown in FIG. 39 to facilitate transitioning the frame 502 between the collapsed and expanded conditions. First part 504 and second part 506 of frame 502 may be coupled together in any manner including ties or a magnetic connection, for example.

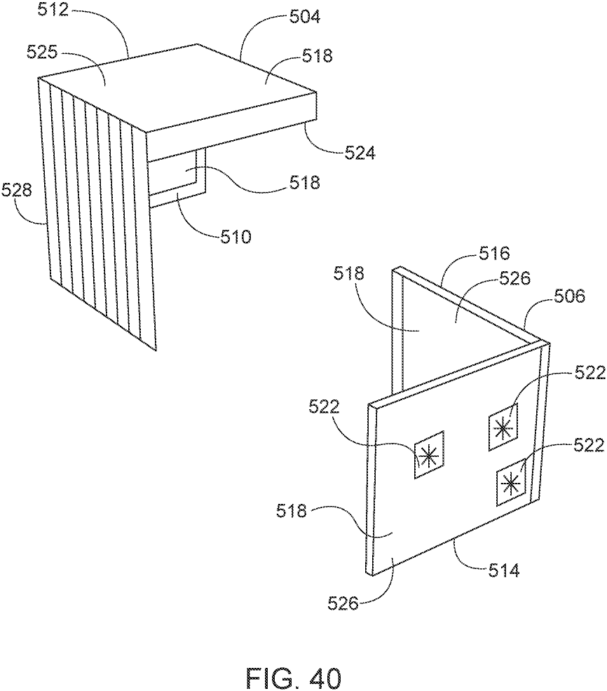

As shown in FIG. 40, a plurality of rigid panels 518 may be attached to the frame 502 and act as a protective layer between the patient 50 to be intubated and the clinician. The panels 518 may be attached in any manner, for example, via known connections such as hook and loop fasteners, snaps, two-sided tape, or other known connection devices. A flap 524, as shown in FIG. 40, may insulate the connections between the separable frame components 504, 506.

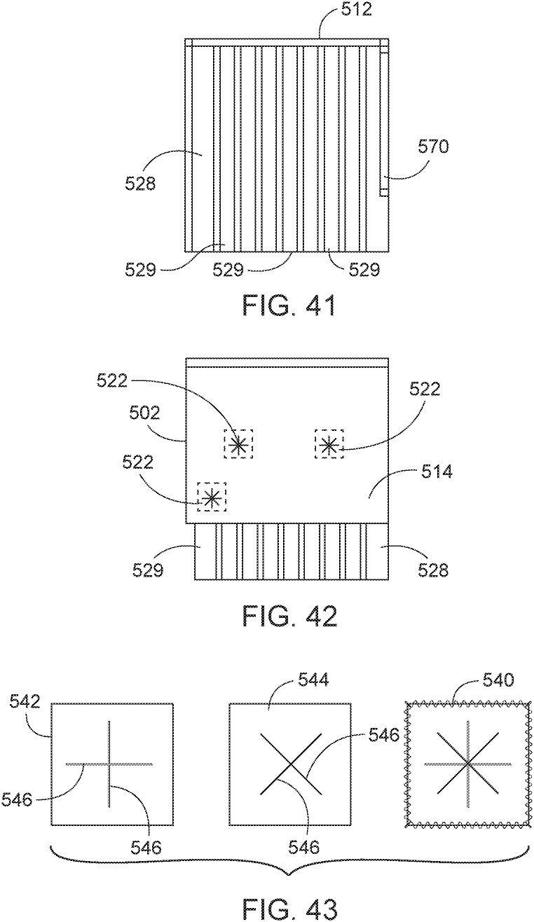

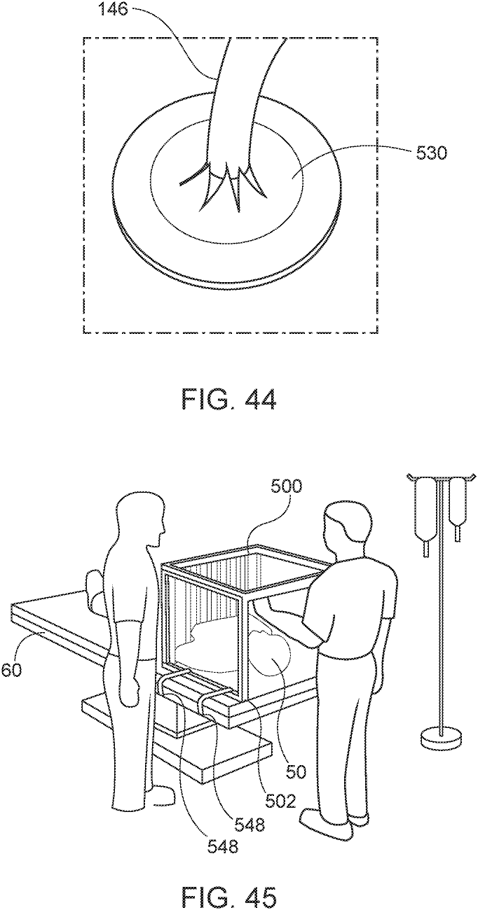

FIGS. 40 and 42 illustrate an exemplary variation of panels 518. As shown, the top cover 525 may be a solid plastic, while the right, left and rear covers 526 may include a plurality of through-openings 522 and, as seen in FIG. 41, the front cover 528, opposite rear cover 526, may include a series of overlapping flaps 529 through which the patient’s head 52 may enter the enclosure 500 or, if enclosure 500 is placed over top of patient 50, flaps 529 may lay across torso 54 of patient 50 lying in a prone position on stretcher or gurney 60. One or more of the covers 526 may include a port 530 (as shown in FIG. 44) through which air may be suctioned during use, or between uses, to remove aspirate (which may contain a virus) from inside enclosure 500.

FIG. 43 illustrates one exemplary cover 540 for a through-opening 522 that includes two sheets 542, 544 bonded together along a periphery of each sheet 542, 544. Each of the sheets 542, 544 includes two cuts 546 extending at 90 degrees to one another, the first sheet 542 and the second sheet 544 being at 45 degrees to one another. The configuration of through-opening 522 formed in this manner provides the cover 540 with a conforming fit around the clinician’s arm (not shown) while inserted through the through-opening 522 and a quick closing of cover 540 after the clinician has removed his/her arm from the interior of enclosure 500.

As shown in FIG. 45, enclosure 500 can be attached to a surgical bed or gurney 60 or rail via one or more straps 548 that are connected to the frame 502.

While different embodiments of enclosures 100-500 are described herein with specific features (e.g., straps 106, 108, through-openings 138, 420, rigid side panels 410, 412, 414, glove 140), while other embodiments are not necessarily described with such features, those skilled in the art will recognize that features described in less than all embodiments can be associated with the remaining embodiments as well.

Also, while enclosures 100-500 are shown to be used with a human patient, as shown in FIG. 7, those skilled in the art will recognize that enclosures 100-500 can also be used for a small animal such that the entire animal can fit inside any of enclosure 100-500.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A containment enclosure comprising:
a frame having:
a rear portion;
a left portion attached to the rear portion;
a right portion attached to the rear portion, opposite from the left portion; and
a top portion attached to the rear portion, the left portion, and the right portion; and
a flexible sheet disposed over at least part of the frame, the flexible sheet covering a front portion.

2. The containment enclosure according to claim 1, further comprising a base portion attached to the left portion and to the right portion.

3. The containment enclosure according to claim 2, wherein the left portion and the right portion are foldable downward toward the base portion.

4. The containment enclosure according to claim 2, wherein the base portion has a through-opening formed therein, the through-opening configured to allow air to be drawn from inside the enclosure.

5. The containment enclosure according to claim 1, wherein the sheet covers the top portion, the left portion, the right portion, and the rear portion.

6. The containment enclosure according to claim 5, wherein the sheet comprises a plurality of vertical strips along the left portion, the right portion, and the rear portion.

7. The containment enclosure according to claim 6, wherein the vertical strips comprise a first row of strips and a second row of strips behind the first row and offset from the first row.

8. The containment enclosure according to claim 1, wherein the frame is of unitary construction.

9. The containment enclosure according to claim 1, wherein the frame comprises a plurality of removably connected pieces.

10. The containment enclosure according to claim 1, wherein the flexible sheet comprises a rectangular central portion defining the front portion and the top portion, a left wing defining the left portion, and a right wing defining the right portion.

11. The containment enclosure according to claim 10, wherein the flexible sheet further comprises a plurality of strips defining the rear portion.

12. The containment enclosure according to claim 1, wherein the flexible sheet has a through-opening formed therethrough.

13. The containment enclosure according to claim 12, further comprising a cover over the through-opening.

14. The containment enclosure according to claim 12, further comprising a glove attached to the flexible material, the glove having a glove opening in communication with the through-opening.

15. The containment enclosure according to claim 1, further comprising at least one strap extending from the left side and configured to attach to the right side.

16. The containment enclosure according to claim 1, further comprising a fan attached to one of the top portion, the left portion, the right portion, and the rear portion, the fan configured to draw air from inside the enclosure.

17. The containment enclosure according to claim 1, further comprising a suction tube attached to one of the top portion, the left portion, the right portion, and the rear portion, the suction tube configured to draw air from inside the enclosure.

18. The containment enclosure according to claim 1, wherein the left portion, the right portion, and the rear portion each comprise a rigid transparent side.

19. The containment enclosure according to claim 18, wherein at least one rigid transparent side has a through-opening, the through-opening being covered by a flexible cover.

20. The containment enclosure according to claim 19, wherein the flexible cover comprises a plurality of pie-shaped pieces.

21. The containment enclosure according to claim 1, wherein the rear portion is hingedly connected to the left portion and forming a first part and wherein the top portion is hingedly connected to the right portion and forming a second part, wherein the first part and the second part comprise separable components.

22. The containment enclosure according to claim 21, wherein the first part is configured to form a first ninety-degree angle and wherein the second part is configured to form a second ninety degree angle such tht the top portion rests on top of the left portion.

23. The containment enclosure according to claim 1, further comprising a base portion releasably attached to the left portion and the right portion.

24. The containment enclosure according to claim 23, wherein each of the left portion and the right portion is hingedly connected to the rear portion.

25. The containment enclosure according to claim 24, wherein the rear portion has a length at least as long as the total sum of the length of the left portion and the length of the right portion.

26. The containment enclosure according to claim 25, wherein the left portion, the right portion, and the rear portion each comprises a rigid transparent panel.

27. The containment enclosure according to claim 28, wherein at least one panel has a through-opening extending therethrough.

28. The containment enclosure according to claim 29, further comprising a cover removably placed over the through-opening.

29. The containment enclosure according to claim 28, further comprising a glove attached to at least one of the panels, the glove having a glove opening in communication with the through-opening.

30. The containment enclosure according to claim 1, wherein the flexible sheet extends at least partially around a lower perimeter of the frame, the flexible sheet being a plurality of vertically hanging strips.

31. The containment enclosure according to claim 30, wherein the flexible material extends over an upper perimeter of the frame, forming a roof.

32. The containment enclosure according to claim 31, wherein the flexible sheet is a single unitary sheet.

33. The containment enclosure according to claim 32, wherein the single unitary sheet comprises:
a. a rectangular main body;
b. a pair of wing portions, each wing portion extending from opposing sides of the main body; and
c. the strips extending from a rear of the main body between the opposing sides.

34. The containment enclosure according to claim 32, comprising an air flow port cut through the flexible sheet.

35. The containment enclosure according to claim 32, wherein each strip overlaps adjacent strips.

36. The containment enclosure according to claim 1, further comprising a sensor incorporated into the sheet.

37. The containment enclosure according to claim 1, further comprising at least one sensor attached to at least one of the rear portion, the left portion, the right portion, and the top portion.
